(19) United States
(12) Patent Application Publication
(10) Pub. No.: US 2014/0364872 A1
(43) Pub. Date: Dec. 11, 2014

(54) TISSUE ANCHOR AND DEPLOYMENT DEVICE FOR SAME

(71) Applicant: LC Therapeutics, Inc., Fremont, CA (US)

(72) Inventors: James Longoria, Sacramento, CA (US); Roy Chin, Pleasanton, CA (US)

(21) Appl. No.: 14/296,374

(22) Filed: Jun. 4, 2014

Related U.S. Application Data
(60) Provisional application No. 61/831,454, filed on Jun. 5, 2013.

(51) Int. Cl. A61B 17/064; A61B 17/068

(52) U.S. Cl. CPC A61B 17/0644; A61B 17/068; A61B 2017/0641
USPC 606/139; 606/151

(57) ABSTRACT

Tissue securing devices and methods for using the same are provided. Also provided are delivery apparatuses and methods for delivering tissue securing devices to target tissue sites, as well kits for practicing the same. The devices, delivery apparatuses, kits and methods find use in a variety of different applications, including anchoring devices or other aspects to tissue.

### CROSS-REFERENCE TO RELATED APPLICATIONS

Pursuant to 35 U.S.C. §119 (e), this application claims priority to the filing date of U.S. Provisional Patent Application Ser. No. 61/831,454 filed Jun. 5, 2013; the disclosure of which application is herein incorporated by reference.

### INTRODUCTION

A variety of surgical tissue fasteners (e.g., anchors) and devices to deploy surgical tissue fasteners are available for medical procedures. Such procedures may include attaching one tissue to another tissue or attaching a device (e.g., an implantable prosthetic device) to tissue within the body of a patient.

One example of a procedure in which a tissue is attached to another tissue is closing a cut or a wound by holding tissues on opposing sides of the cut or wound together. Such a procedure may facilitate healing of the cut or wound. Fasteners that may be applied in closing a cut or wound to hold respective tissues in a desired position include staples, sutures, clips and/or other surgical fasteners.

Another example of a procedure in which a tissue is attached to another tissue is a surgical procedure requiring the attachment of two tissues to facilitate the operation of one or more body systems. For example, gastroesophageal reflux disease (GERD) may be treated by a surgical procedure known as fundoplication. Fundoplication is meant to alleviate acid reflux in patients whose lower esophageal sphincter (LES) has been impaired, for example, as a result of GERD. In fundoplication, the gastric fundus of the stomach is plicated around the lower end of the esophagus and fastened in place. The fundus is held in place by using a surgical fastener to attach the fundus to itself and to the esophagus such that the fundus completely encircles the esophagus. Plicating the gastric fundus around the lower end of the esophagus and fastening it in place improves the closing ability of the lower esophageal sphincter and thereby alleviates symptoms of GERD. A specific example of a fastener configured for use in a fundoplication procedure is the device provided by U.S. Pat. No. 7,811,295 and is shown in FIGS. 1A and 1B.

An example of a procedure in which a device is introduced into the body of a patient and attached with a surgical fastener to tissue within the body of a patient is the implantation of surgical mesh material. Surgical mesh material has various uses in surgery, such as supporting, immobilizing, and/or positioning one or more body organs or other tissues. In a typical circumstance, the mesh is secured to body tissues at two or more locations by using surgical fasteners or suturing. For example, in inguinal hernia surgery, a surgical mesh is fastened to the abdominal wall at one or more locations in order to reinforce the abdominal wall. Introduction of surgical mesh material using surgical tissue fasteners has also been used in the treatment of urinary incontinence, uterine prolapse and hernia. A specific example of a fastener configured for use in surgical mesh attachment is the device provided by U.S. Pat. No. 6,447,524 and is shown in FIG. 2.

### SUMMARY

Tissue securing devices and methods for using the same are provided. Also provided are delivery apparatuses and methods for delivering tissue securing devices to target tissue sites, as well kits for practicing the same. The devices, delivery apparatuses, kits and methods find use in a variety of different applications, including anchoring devices or other aspects to tissue.

### BRIEF DESCRIPTION OF THE FIGURES

FIG. 1A provides a lateral view of a tissue fastening device of the prior art. FIG. 1B provides a top view of a tissue fastening device of the prior art.

FIG. 2 provides a side view of a tissue fastening device of the prior art.

FIG. 3 provides a view of a tissue securing device in an un-deployed configuration in accordance with an embodiment of the invention.

FIGS. 4A & 4B provide views of a tissue securing device in a deployed configuration in accordance with an embodiment of the invention.

FIG. 5A provides a diagram of a tissue securing device in an un-deployed configuration and in a deployed configuration according to a first embodiment of the present disclosure. FIG. 5B provides a diagram of a tissue securing device in an un-deployed configuration and in a deployed configuration according to another embodiment of the present disclosure. FIG. 5C provides a view of a tissue securing device in a deployed configuration according to an embodiment of the invention.

FIG. 6A provides a view of a tissue site (e.g., a target tissue site). FIG. 6B provides a view of a tissue site (e.g., a target tissue site), an implantable device and a portion of a delivery apparatus in accordance with an embodiment of the invention. FIG. 6C provides a view of a tissue site (e.g., a target tissue site) and an implantable device according to embodiments of the present disclosure.

FIG. 7 provides a view of a delivery apparatus according to embodiments of the present disclosure.

FIG. 8 provides a view of a portion of a delivery apparatus according to embodiments of the present disclosure.

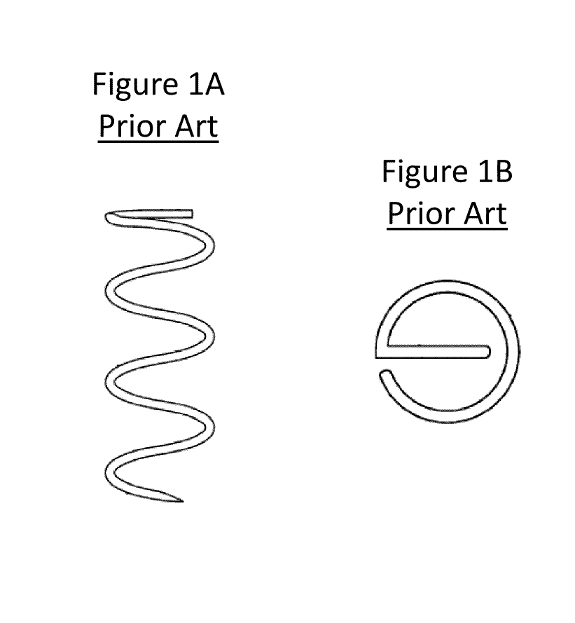

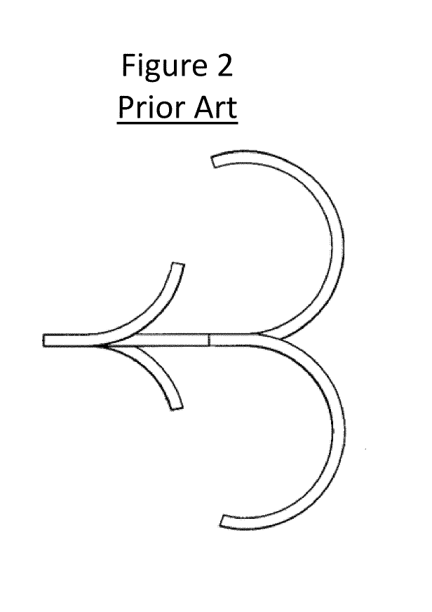

### DEFINITIONS

As used herein, the term “securing” refers to affixing, fastening, anchoring or attaching one aspect (e.g., tissue and/or device, such as a surgical device) to another. Securing may be for a period of time such as for a number of days, weeks, months, years, and/or for at least the remaining lifetime of a subject. When an aspect is secured, it may, for example, be retained at the same position or substantially at the same position (e.g., a position within the body of a subject) for a time period, such as a for a period of days, weeks, months, years and/or for at least the remaining lifetime of a subject.

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform an annuloplasty procedure on a human subject, it is to be understood that the subject devices and methods may also be carried-out to perform an annuloplasty procedure on other subjects (that is, in “non-human subjects”).

The present disclosure provides embodiments of devices (e.g., tissue securing devices) which are implantable. As used herein, the terms “implantable”, “implanted” and “implanting” refer or relate to the characteristic of the ability of an aspect to be placed (e.g., surgically introduced) into a physiological site (e.g., a site within the body of a subject) and maintained for a period of time without substantial, if any, impairment of function. As such, once implanted in or on a body, the aspects do not deteriorate in terms of function, e.g., as determined by ability to perform effectively as described herein, for a period of 2 days or more, such as 1 week or more, 4 weeks or more, 6 months or more, or 1 year or more, e.g., 5 years or more, and/or for the remaining lifetime or expected remaining lifetime of the subject or more. Implantable aspects may also be aspects that are configured (e.g., dimensioned and/or shaped) to fit into a physiological site (e.g., a site within the body of a subject). For example, in certain embodiments, an implantable aspect may have a longest dimension, e.g., length, width or height, ranging from 0.05 mm to 500 mm, such as from 0.2 mm to 250 mm, including from 0.5 mm to 200 mm, e.g., 1 mm to 100 mm. Implanting may also include securing an implanted object (e.g., a prosthetic device) to one or more tissues within the body of the subject. Additionally, implanting may, in some instances, include all of the surgical procedures (e.g., cutting, suturing, sterilizing, etc.) necessary to introduce one or more objects into the body of a subject.

Geometrical terms are also used to describe the disclosed aspects throughout the specification. As such, the term “plane” as used herein refers to a flat surface that is infinitely large with zero thickness, unless a particular thickness is otherwise specified or it can be inferred that the plane has a particular thickness (e.g., the thickness of an arm). Additionally, the term “longitudinal” as used herein refers to the characteristic of being associated with (e.g., placed or running along) a length (e.g., a straight or curved length) or lengthwise dimension.

As used herein, the term “vertical” refers to the direction extending directly upward and/or directly downward. For example, a vertical direction may be the direction that extends through the uppermost point of a spherical object resting on a flat surface (e.g., a horizontal surface) through the lowermost point of the spherical object. Also, the term “horizontal”, as used herein, refers to a direction that is perpendicular to the vertical direction.

The devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Furthermore, the definitions and descriptions provided in one or more (e.g., one, two, three, four, or five, etc.) sections of this disclosure (e.g., the “Devices” and/or “Methods” and/or “Kits” sections below) are equally applicable to the devices, methods and aspects described in the other sections.

### DETAILED DESCRIPTION

Tissue securing devices and methods for using the same are provided. Also provided are delivery apparatuses and methods for delivering tissue securing devices to target tissue sites, as well kits for practicing the same. The devices, delivery apparatuses, kits and methods find use in a variety of different applications, including anchoring devices or other aspects to tissue.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Devices

As summarized above, aspects of the invention include implantable tissue securing devices. Aspects of the invention further include apparatuses for delivering the tissue securing devices to target tissue sites. Each of these aspects of the invention is now described further in greater detail.

### Tissue Securing Devices

Embodiments of the disclosed devices include implantable tissue securing devices. Such devices may be configured to secure one aspect (e.g., tissue and/or device, such as a prosthetic device) to another. For example, one or more (e.g., two, three, four, five, etc.) tissue securing devices may be configured to attach a surgical device (e.g., a surgical device, such as an annuloplasty ring, a surgical mesh, etc.) to one or more tissues within the body of a subject. Tissue securing devices may also, for example, be configured to attach one type of tissue (e.g., tissue within the body of a subject) to another or to another portion of the same type of tissue.

In various embodiments, a tissue securing device has a body having a first arm (e.g., a proximal arm, such as a proximal deployable arm) at a first end (e.g., a proximal end) and a second arm (e.g., a distal arm, such as a distal deployable arm) at a second end (e.g., a distal end). The body of a tissue securing device may also have an elongated linear structure located between the proximal and distal arms of the device.

In some aspects of tissue securing devices, elongated linear structures form a body of one or more materials extending longitudinally in a proximal-distal direction. Elongated linear structures may have a center-most line (e.g., a straight line) extending through the center-most portion of the structures (e.g., the center-most points of each transverse cross section of the structure along the longitudinal length of the structure). In some aspects, elongated linear structures may have an axis (e.g., an axis of symmetry and/or a long axis) extending (e.g., extending longitudinally) through and along the length of an elongated linear structure. In certain aspects, the center-most line extending through an elongated linear structure and/or the axis of an elongated linear structure may be continuous with (e.g., connect to) the center-most line of another portion (e.g., the remaining portion of the body or one or more arms) of the tissue securing device.

In some embodiments of devices including elongated linear structures, the diameter, radius, thickness, peripheral circumference and/or cross sectional area (e.g., the area of the device within a plane, such as a cross-sectional plane that is perpendicular to or substantially perpendicular to the axis) of an elongated linear structure along the length of its axis remains the same. In some embodiments, such dimensions increase and/or decrease along the length of the axis of an elongated linear structure from a first to a second end of the elongated linear structure or from a second end to a first end of the elongated linear structure.

In certain versions of elongated linear structures of the subject devices, a cross section (e.g., a profile of the elongated linear structures within a plane, such as a plane, such as a cross-sectional plane, that is perpendicular to the center-most line or axis) of an elongated linear structure at a point along the elongated linear structure is shaped as a circle (e.g., forming a circular cross-section), semi-circle, oval, rectangle (e.g., forming a rectangular cross-section), square, triangle, polygon, quadrilateral, or any combination thereof. Such a cross section of an elongated linear structure may also be curvilinear. Additionally, a cross section of an elongated linear structure may also have the same shape and/or size as one or more (e.g., one, two, three, four, etc.) arms (e.g., arms attached to and/or continuous with the elongated linear structure) of a tissue securing device, or portions of such arms.

As discussed above, embodiments of the disclosed tissue securing devices include devices in which the elongated linear structure is positioned between two or more (e.g., two, three, four, etc.) arms of the device. In some aspects, devices include an elongated linear structure positioned between a first set of one or more arms (e.g., one, two, three, four, etc.) at a first end (e.g., a proximal end) and a second set of one or more arms (e.g., one, two, three, four, etc.) arms at a second end (e.g., a distal end). In some embodiments, a first end (e.g., a distal end) of an elongated linear structure is continuous with and/or connected to an end of one or more arms (e.g., deployable arms) and/or a second end (e.g., a proximal end) of an elongated linear structure is continuous with and/or connected to an end of one or more arms. In some aspects, a first end of an elongated linear structure is oriented in a proximal direction and/or a second end of an elongated linear structure is oriented in a distal direction.

As noted above, tissue securing device or a portion thereof (e.g., a body) may have one or more (e.g., one, two, three, four, five, etc.) arms. In various embodiments of the disclosed devices, the arm or arms of the devices are one or more (e.g., one, two, three, four, five, etc.) structures which may be composed of one or more materials (e.g., any of the materials described herein) and which extend from another portion of a tissue securing device, such as an elongated linear structure and/or another arm.

Each arm may have a first end (e.g., a proximal or distal end) and a second end (e.g., a proximal or distal end) opposite the first. In some embodiments, an end (e.g., a first end) of an arm is continuous with (e.g., composed of a continuous piece of one or more materials with) or attached to the distal or proximal end of one of the other portions of a tissue securing device, such as the elongated linear structure of a device. In some embodiments of tissue securing devices, an end (e.g. a second end) of an arm is sharpened (e.g., configured to easily pierce and/or move through tissue) to a point or blade.

In certain embodiments of the subject devices, an arm of a device includes a center-most line (e.g., a straight and/or curved line) extending through the center-most portion of each arm (e.g., the center-most points of each transverse, e.g., perpendicular or substantially perpendicular, cross section of the arm along the longitudinal length of an arm). In some aspects, the center-most line extending through an arm may be continuous with (e.g., connect to) the central axis (e.g., the central longitudinal axis and/or long axis) of another portion (e.g., the remaining portion of the body or the elongated linear structure of the body) of the tissue securing device. In aspects in which the center-most line of an arm or a portion thereof is curved, the curved portion may have two or more radii of curvature lying within a plane.

In some embodiments of arms having a center-most line, the diameter, radius, thickness, peripheral circumference and/or cross sectional area (e.g., the area of the device within a plane, such as a cross-sectional plane that is perpendicular to or substantially perpendicular to the center-most line or a tangent line thereof) of the arm along the length of the line remains the same. In some embodiments, such dimensions increase and/or decrease along the length of the center-most line of an arm from a first to a second end of the arm or from a second end to a first end of the arm.

In certain versions of arms of the disclosed devices, a cross section (e.g., a profile of the arm within a plane, such as a plane, which may be a cross-sectional plane, that is perpendicular to or substantially perpendicular to the center-most line or a tangent line thereof) of an arm at a point along the arm is shaped as a circle (e.g., forming a circular cross-section), semi-circle, oval, rectangle (e.g., forming a rectangular cross-section), square, triangle, polygon, quadrilateral, or any combination thereof. Such a cross section of an arm may also be curvilinear. A cross section of one or more (e.g., one, two, three, four, five, etc.) arms (e.g., one or more arms at a proximal end of a device) of a device may have the same or different size and shape as that of one or more (e.g., one, two, three, four, five, etc.) other arms (e.g., one or more arms at a distal end of a device) of the device.

As mentioned above, certain embodiments of the subject devices include deployable arms. As used herein, the term “deployable” refers to the characteristic of having the ability to move (e.g., deploy) or be moved (e.g., deployed) from a first (e.g., initial and/or un-deployed) position (e.g., a first spatial position) to a second (e.g., consequent and/or deployed) position (e.g., a second spatial position) that is different from the first position and/or from a second position to a first position. Deployment may be irreversible or reversible. Additionally, the terms “first” and “second” are used herein in describing positions of one or more arms to distinguish between different positions and are irrespective of the order in which the configurations or associated events they are describing may occur. For example, in some embodiments, deployable aspects described herein may be placed in a deployed position before being placed in an un-deployed position or placed in an un-deployed position before being placed in a deployed position.

In some aspects, tissue securing devices may be un-deployed (i.e., have an un-deployed configuration) when one or more (e.g., two, three, four, five, etc.) arms of the device are un-deployed. Likewise, in various aspects, tissue securing devices may be deployed (i.e., have a deployed configuration) when one or more (e.g., two, three, four, five, etc.) arms of the device are deployed.

Embodiments of deployable arms include arms having a first (e.g., un-deployed) configuration, in which an arm is in a first position (e.g., a first spatial position) and a second (e.g., deployed) configuration, in which an arm is in a second position (e.g., a second spatial position). In certain embodiments, deployable arms are movable between an un-deployed and deployed configuration.

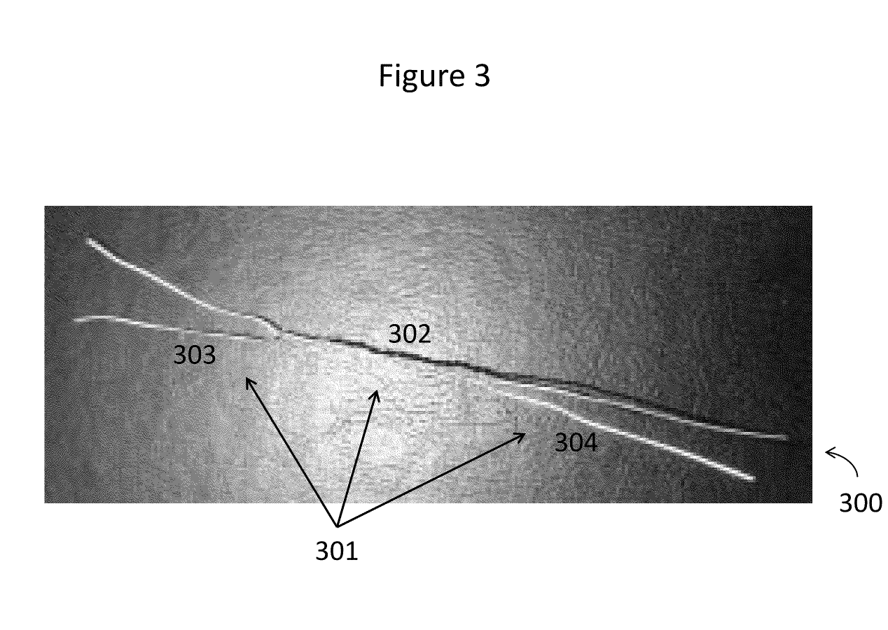

FIG. 3 provides a view of a tissue securing device in an un-deployed configuration 300 in accordance with embodiments of the invention. FIG. 3 specifically shows a body 301 of a tissue securing device as well as an elongated linear structure 302 thereof. Additionally, FIG. 3 shows a pair of proximal arms including a proximal deployable (e.g., in an un-deployed configuration) arm 303 as well as a pair of distal arms including a distal deployable (e.g., in an un-deployed configuration) arm 304.

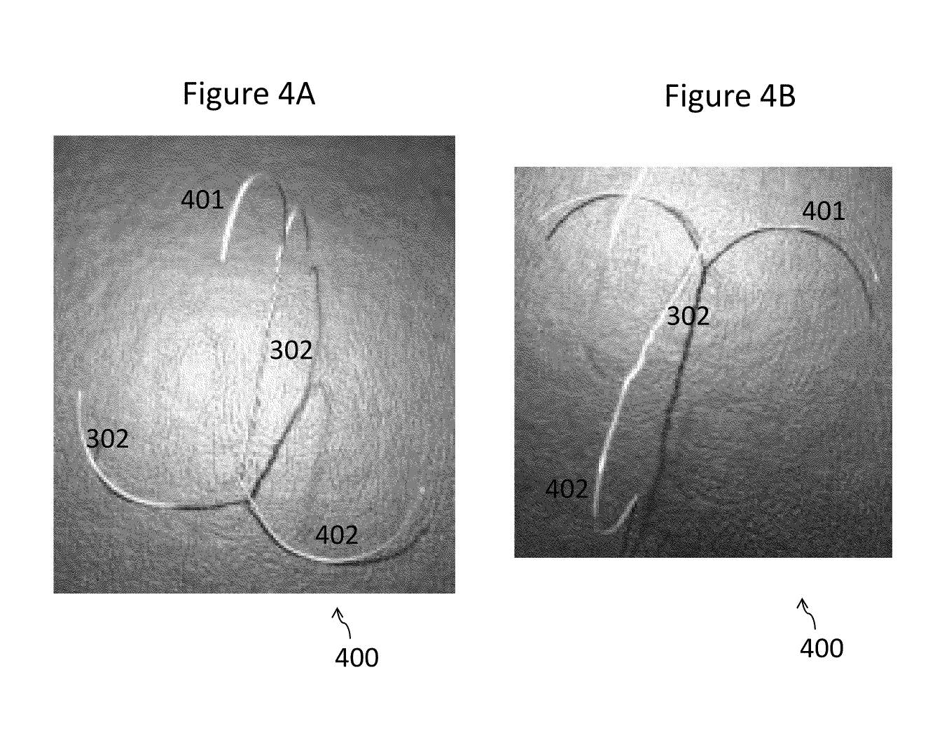

FIG. 4A provides a depiction of a tissue securing device in a deployed (e.g., non-coplanar) configuration 400 in accordance with an embodiment of the invention. In addition, FIG. 4A illustrates a pair of proximal arms including a proximal deployed (i.e., in a deployed configuration) arm 401. FIG. 4A also illustrates a pair of distal arms including a distal deployed (i.e., in a deployed configuration) arm 402. FIG. 4A also specifically shows an elongated linear structure 302 of the tissue securing device thereof.

Variations of the disclosed embodiments include devices having one or more (e.g., one, two, three, four, five, etc.) portions (e.g., deployable portions, such as deployable arms) which have a bias to remain in and/or return to a certain configuration (e.g., a deployed position). For example, various embodiments of devices include portions (e.g., one or more arms) which are biased (e.g., strongly biased) to return to a deployed position after they are moved to an un-deployed position from a deployed position. In other words, certain embodiments of devices include portions (e.g., one or more arms) having a characteristic tendency (e.g., an ongoing characteristic tendency) to return to their original shape after being deformed from their original shape. One or more (e.g., one, two, three, four, five, etc.) portions (e.g., deployable portions, such as deployable arms) of certain embodiments of the disclosed devices may have a such bias because the one or more portions are composed of a shape memory material, which is described in greater detail below.

In some embodiments, the center-most line of an arm when the arm is in a first (e.g., un-deployed) position is in the same plane as the center-most line of the arm when the arm is in a second (e.g., deployed) position. In various embodiments, the center-most line of an arm when the arm is in a first (e.g., un-deployed) position remains in the same plane as the center-most line of the arm when the arm is in a second (e.g., deployed) position as the arm is moved between the first position and the second position (e.g., from the first position to the second position or from the second position to the first position).

In various embodiments, an arm in a first (e.g., un-deployed) position is in the same plane (e.g., a plane having the thickness of the arm) as the arm when the arm is in a second (e.g., deployed) position. In various embodiments, an arm initially positioned in a first (e.g., un-deployed) position remains in the same plane (e.g., a plane having the thickness of the arm) as the arm moves between the first position and the second position (e.g., from the first position to the second position or from the second position to the first position).

In some embodiments of the subject devices, the center-most line of each of two or more (e.g., two, three, four, five, etc.) arms when one or more (e.g., one, two, three, four, five, etc.) of the arms are in a first (e.g., un-deployed) position are in the same plane as the center-most line of each of the two or more (e.g., two, three, four, five, etc.) arms when one or more (e.g., one, two, three, four, five, etc.) of the arms are in a second (e.g., deployed) position. In various embodiments, the center-most line of each of two or more (e.g., two, three, four, five, etc.) arms when one or more (e.g., one, two, three, four, five, etc.) of the arms are in a first (e.g., un-deployed) position remain in the same plane as the center-most line of each of the two or more (e.g., two, three, four, five, etc.) arms when one or more (e.g., one, two, three, four, five, etc.) of the arms is moved between the first position and the second position (e.g., from the first position to the second position or from the second position to the first position).

In various embodiments, each of two or more (e.g., two, three, four, five, etc.) arms when one or more (e.g., one, two, three, four, five, etc.) of the arms are in a first (e.g., un-deployed) position are in the same plane (e.g., a plane having the thickness of the arms) as the two or more (e.g., two, three, four, five, etc.) arms when one or more (e.g., one, two, three, four, five, etc.) of the arms are in a second (e.g., deployed) position. In various embodiments, each of two or more (e.g., two, three, four, five, etc.) arms when one or more (e.g., one, two, three, four, five, etc.) of the arms are in a first (e.g., un-deployed) position remain in the same plane (e.g., a plane having the thickness of the arms) as the two or more (e.g., two, three, four, five, etc.) arms when one or more (e.g., one, two, three, four, five, etc.) of the arms is moved between the first position and the second position (e.g., from the first position to the second position or from the second position to the first position).

Where appropriate, the subject devices include one or more portions (e.g., one or more arms, such as deployable arms) in a non-coplanar configuration. As used herein, the term “coplanar” refers to the characteristic of two or more aspects (e.g., one or more arms, such as deployable arms, and/or other portions of a body of a tissue securing device, such as an elongated linear structure) and/or the center-most line thereof (e.g., the axis, such as the axis of symmetry) lying within the same plane (e.g., a plane having the thickness of an arm). Similarly, the term “substantially coplanar”, as used herein, refers to the characteristic of two or more aspects (e.g., arms) substantially lying within (e.g., having a majority portion, such as more than 50%, 75%, 90%, 95%, or nearly all of a portion lying within) the same plane (e.g., a plane having the thickness of an arm). Accordingly, the term “non-coplanar” refers to the characteristic of two or more aspects (e.g., arms) and/or the center-most line thereof (e.g., the axis, such as the axis of symmetry) not lying entirely within the same plane (e.g., a plane having the thickness of an arm). Likewise, the term “substantially non-coplanar”, as used herein, refers to the characteristic of two or more aspects (e.g., arms) substantially not lying within (e.g., having a majority portion, such as more than 50%, 75%, 90%, 95%, or nearly all of a portion not lying within) the same plane (e.g., a plane having the thickness of an arm).

Embodiments of the subject devices include devices having a first arm (e.g., a proximal arm, such as a deployable arm at a proximal end of the body of a tissue securing device) and a second arm (e.g., a distal arm, such as a deployable arm at a distal end of the body of a tissue securing device) wherein, upon deployment of the first and second arms, the first and second arms are in a non-coplanar configuration or a substantially non-coplanar configuration.

Tissue securing devices, in some aspects, may include one or more (e.g., first and second) arms (e.g., deployable arms) at the proximal and/or distal end of the body of the device or a portion thereof (e.g., an elongated linear structure). In certain embodiments of tissue securing devices, such as devices having first and second deployable arms at the proximal end of the body, upon deployment of the first and second proximal deployable arms, the arms are in a planar configuration. As used herein, “planar configuration” refers to the characteristic (e.g., spatial characteristic) of falling within or substantially falling within a single plane, (e.g., a plane having the thickness of one or more various aspects). In certain embodiments of tissue securing devices, such as devices having first and second deployable arms at the distal end of the body, upon deployment of the first and second distal deployable arms, the arms are in a planar configuration.

In certain embodiments of tissue securing devices, such as devices having first and second deployable arms at the proximal end of the body and first and second deployable arms at the distal end of the body, upon deployment of the first and second proximal deployable arms at the proximal end of the body and the first and second proximal deployable arms at the proximal end of the body, the arms are in a non-coplanar configuration (e.g., the arms at the distal end are in a plane, such as a plane having the thickness of the arms which is orthogonal and/or perpendicular to a plane in which arms at the proximal end are in). In such an embodiment, the plane of the arms at the distal end may be oriented at angle, e.g., to 10° or greater, such as 20° or greater, 30° or greater, 40° or greater, 45° or greater, 50° or greater, 60° or greater, 70° or greater, 80° or greater, 90° or greater, 120° or greater, 135° or greater, 160° or greater, up to 180° (e.g., 45°, 90°, 135°) with respect to the plane of the arms at the proximal end.

In some versions of the disclosed devices, one or more (e.g., one, two, three, four, etc.) proximal and/or distal arms deploy (e.g., deploy within a plane) orthogonally from the body of the device. In some embodiments, one or more (e.g., one, two, three, four, etc.) proximal and/or distal arms deploy orthogonally with respect to each other and/or with respect to another portion of the device (e.g., one or more other arms and/or an elongated linear structure). In some aspects of the disclosed devices, one or more (e.g., one, two, three, four, etc.) proximal and/or distal arms deploy (e.g., deploy within a plane) at an angle (e.g., 45°, 90°, 135°) from the body of the device. In some embodiments, one or more (e.g., one, two, three, four, etc.) proximal and/or distal arms deploy at an angle (e.g., 45°, 90°, 135°) with respect to each other and/or with respect to another portion of the device (e.g., one or more other arms and/or an elongated linear structure).

FIG. 4B illustrates a tissue securing device in a deployed configuration in accordance with an embodiment of the invention. In addition, FIG. 4B illustrates a pair of proximal arms including a proximal deployed arm 401. FIG. 4B also illustrates a pair of distal arms including a distal deployed arm 402. FIG. 4B shows the pair of proximal arms (including proximal deployed arm 401) deployed in an orthogonal configuration with respect to the pair of distal arms (including distal deployed arm 402). FIG. 4B also specifically shows an elongated linear structure 302 of the tissue securing device.

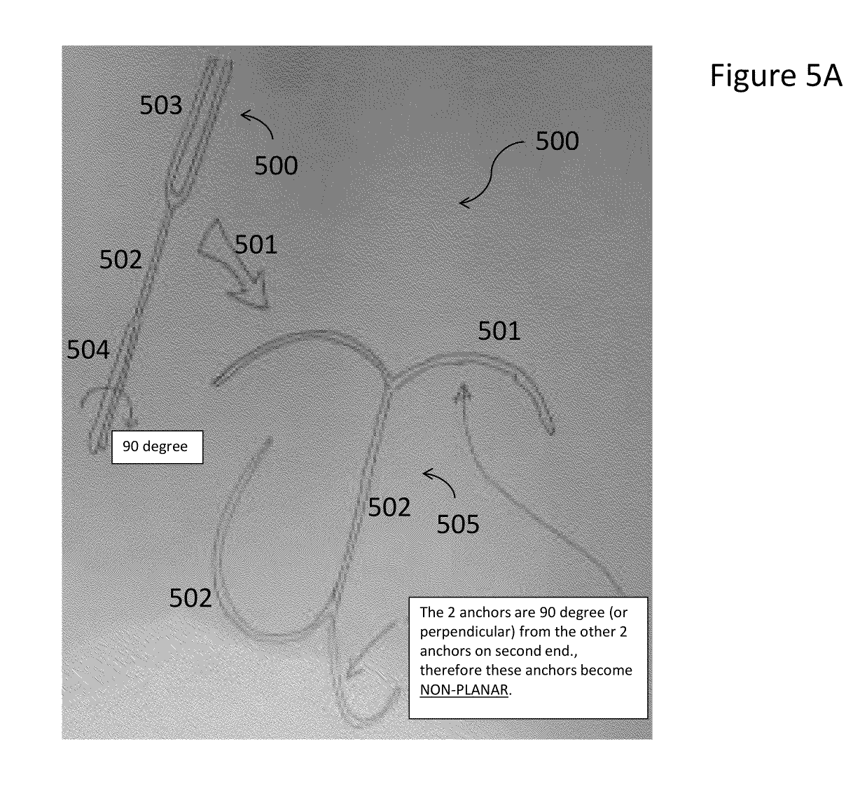

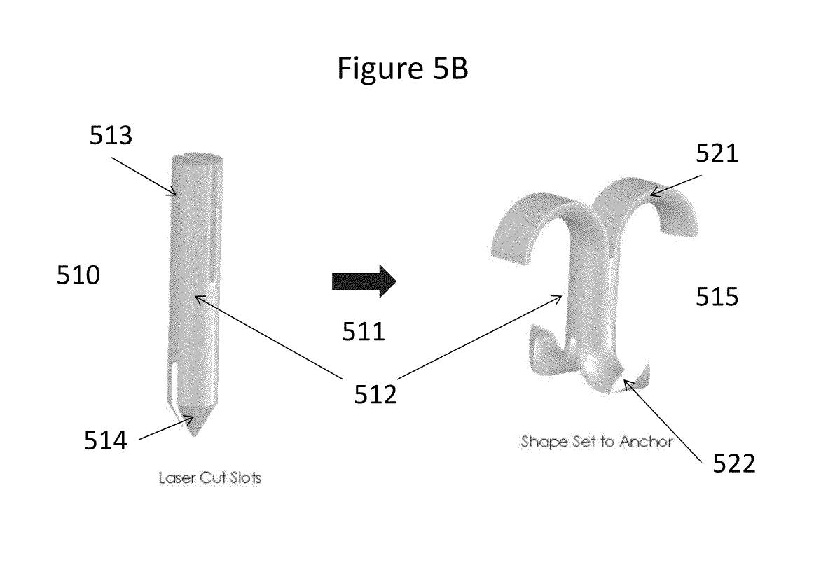

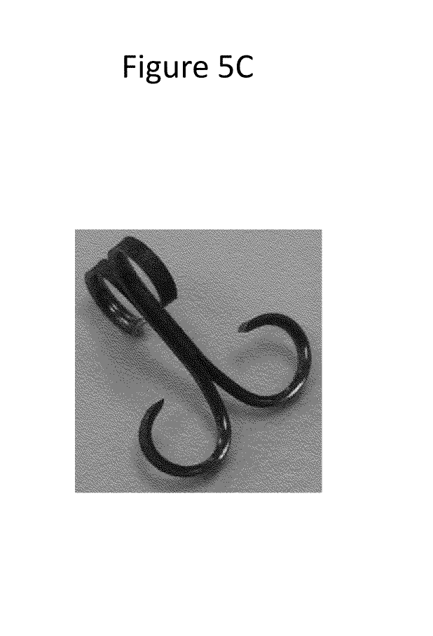

FIG. 5A provides a view of deployment 500 of a fastener from an un-deployed configuration to a deployed configuration in accordance with an embodiment of the invention. FIG. 5A includes an illustration of a tissue securing device in an un-deployed configuration 500 as well as an illustration of a tissue securing device in a deployed configuration 505 in accordance with an embodiment of the invention. The transition between an un-deployed configuration to a deployed configuration is shown in FIG. 5A by an arrow 501. FIG. 5A specifically shows an elongated linear structure 502 of a tissue securing device. Additionally, FIG. 5A shows a pair of proximal arms including a proximal deployable (i.e., in an un-deployed configuration) arm 503 as well as a pair of distal arms including a distal deployable (i.e., in an un-deployed configuration) arm 504. Furthermore, FIG. 5A illustrates a pair of proximal arms including a proximal deployed (i.e., in a deployed configuration) arm 501. FIG. 5B also illustrates a pair of distal arms including a distal deployed (i.e., in a deployed configuration) arm 502. Furthermore, FIG. 5A depicts the pair of proximal arms (including proximal deployed arm 501) deployed in an orthogonal configuration with respect to the pair of distal arms (including distal deployed arm 502).

FIG. 5B provides a view of un-deployed and deployed configurations of a device according to another embodiment. FIG. 5B includes an illustration of a tissue securing device in an un-deployed configuration 510 as well as an illustration of a tissue securing device in a deployed configuration 515 in accordance with an embodiment of the invention. The transition between an un-deployed configuration to a deployed configuration is shown in FIG. 5B by an arrow 511. FIG. 5B specifically shows an elongated linear structure 512 of a tissue securing device. Additionally, FIG. 5B shows a pair of proximal arms including a proximal deployable (i.e., in an un-deployed configuration) arm 513 as well as a pair of distal arms including a distal deployable (i.e., in an un-deployed configuration) arm 514. Furthermore, FIG. 5A illustrates a pair of proximal arms including a proximal deployed (i.e., in a deployed configuration) arm 521 and a pair of distal arms including a distal deployed (i.e., in a deployed configuration) arm 522. Furthermore, FIG. 5B depicts the pair of proximal arms (including proximal deployed arm 521) deployed in an orthogonal configuration with respect to the pair of distal arms (including distal deployed arm 522). As can be seen in the un-deployed configuration 510, the device is a tubular device of a shape memory material having orthogonally positioned slots (which may be laser cut slots). In addition, the distal end of the device has a pointed (i.e., conical) shape to so that it may pass through tissue prior to deployment of the arms. As a result, as seen in deployed configuration 515, the distal arms have pointed ends and the proximal arms have blunt ends. FIG. 5C provides a picture of a device in a deployed configuration fabricated from a shape memory material, e.g., as illustrated in FIG. 5B.

Embodiments of the subject devices include devices having two or more (e.g., two, three, four, five, etc.) arms (e.g., proximal arms, such as deployable arms) at a proximal end of the body of a tissue securing device and two or more (e.g., two, three, four, five, etc.) arms (e.g., distal arms, such as deployable arms) at a distal end of the body of a tissue securing device. In some embodiments of such devices, upon deployment of at least one of the arms at the proximal end of the body of the device and deployment of at least one of the arms at the distal end of the body of the device, the deployed arms at the proximal end of the body of the device are in a non-coplanar configuration or a substantially non-coplanar configuration with respect to the at least one of the deployed arms at the distal end of the body of the device.

In certain versions of the disclosed tissue securing devices, the devices include one or more arms or center-most lines thereof that, when in one configuration (e.g., an un-deployed configuration) have a linear shape or a substantially linear shape. In such a configuration, one end, such as a distal end (e.g., a sharp end) of one or more (e.g., one, two, etc.) arms may be configured to pierce and/or enter and/or move through tissue in a direction that is, at least in part, for example, substantially parallel to the axis of the elongated linear structure of the device (e.g., a distal direction).

In various embodiments of the subject tissue securing devices, the devices include two or more arms (e.g., arms that are both or all at a proximal and/or distal end of a device) that, when in one configuration (e.g., an un-deployed configuration) extend or have a portion that extends from an elongated linear structure of the device at an angle (e.g., an acute angle) with respect to the other one or more arms or portion of one or more arms. In some variations, the disclosed devices include one or more arms that extend from one or more ends of an elongated linear structure of the body of a device at an angle (e.g., an obtuse angle) with respect to the elongated linear structure or an angle (e.g., an acute angle) with respect to the axis thereof.

The disclosed tissue securing devices, in certain instances, include one or more (e.g., one, two, three, four, or five, etc.) arms (e.g., at least one or two arms at a distal end of a tissue securing device body and/or at least one or two arms at a proximal end of a tissue securing device body) that, when in one configuration (e.g., a deployed configuration) assume a curvilinear configuration (i.e., have a shape that is curvilinear) or have one or more portions which assume a curvilinear configuration. In some versions of arms which assume a curvilinear configuration or have a portion which assume a curvilinear configuration, the curvilinear configuration may be an arc configuration (i.e., shaped as an arc and/or have an arcing portion). Additionally, the disclosed devices, in various embodiments, include one or more (e.g., one, two, three, four, or five, etc.) arms (e.g., at least one or two arms at a distal end of a tissue securing device body and/or at least one or two arms at a proximal end of a tissue securing device body) that, when in one configuration (e.g., a deployed configuration) are shaped as a partial ring and/or shaped substantially like a capital letter “C”.

In some aspects of the subject devices having one or more arms having a curvilinear configuration (e.g., an arc configuration), the curved portions may have one or more (e.g., two or more) radii of curvature lying within a plane. A given radius of curvature may vary, ranging in some instances from 0.1 mm to 10 mm, such as 0.5 mm to 5 mm. Such a plane may be the same plane as that running through the center-most line of one or more (e.g., two or more) arms, (e.g., such as the arm having the curve) of the device and/or the axis of an elongated linear structure of the device. Accordingly, a plane having within it one or more (e.g., two or more) radii of curvature of an arm may have any of the other characteristics of any of the planes described herein. In some versions of the disclosed devices having one or more arms having an arc configuration, the arm may be within a plane (e.g., a plane having the thickness of the arm). Such a plane may have any of the characteristics of any of the planes described herein.

In various embodiments of the disclosed tissue securing devices, the devices have a body or one or more portions thereof (e.g., one or more arms, e.g., deployed arms and/or un-deployed arms, and/or an elongated linear structure) having a length (e.g., a length from a proximal end to a distal end) ranging from 1 mm to 30 mm, such as from 1 mm to 20 mm, including from 1 mm to 10 mm. For example, in various embodiments of the subject tissue securing devices, the devices have an elongated linear structure (and/or a center-most line thereof) having a length (e.g., a length from a proximal end to a distal end and/or a length along the axis) ranging from 1 mm to 30 mm, such as from 1 mm to 20 mm, including from 5 mm to 15 mm. Furthermore, and also for example, in various embodiments of the disclosed devices, the devices have an arm (and/or a center-most line thereof) having a length ranging from 1 mm to 20 mm, such as from 1 mm to 10 mm, including from 1 mm to 5 mm. Additionally, in certain embodiments, tissue securing devices have a body or one or more portions thereof that are dimensioned to fit through an opening (e.g., a circular opening) having a diameter ranging from 1 mm to 10 mm, such as 1 mm to 5 mm or 1 mm to 3 mm. In various embodiments, tissue securing devices include a body or one or more portions thereof that are dimensioned to fit through a circular opening having a diameter of 3 mm or less. Furthermore tissue securing devices may have a body or one or more portions thereof that are dimensioned to fit into an opening (e.g., a tubular opening) having a volume ranging from 0.1 mm³ to 10 mm³, e.g., 1 mm³ to 5 mm³, including from 1 mm³ to 2 mm³.

In versions of the disclosed devices in which the devices include one or more (e.g., one, two, three, four, or five, etc.) arms (e.g., deployed arms) having a curvilinear configuration (e.g., an arc configuration), the curved portions of one or more of the arms may curve outward from an elongated linear structure of a tissue securing device in a first direction that is at least partially along the axis of the elongated linear structure (e.g., a distal direction) and may also curve in a direction that is at least partially along the axis of the elongated linear structure in a second direction which is at least partially in the opposite direction than the first direction. In some cases, one or more (e.g., one, two, three, four, or five, etc., or all) arms of a tissue securing device are shaped as a hook (e.g., a hook having a sharpened or non-sharpened end).

In some aspects of the disclosed devices having two or more arms (e.g., one or more arms at a distal end of a device and one or more arms at a proximal end of the device) having a curvilinear configuration (e.g., an arc configuration), the curved portions of the two or more arms (e.g., two sets of two arms) may orient (e.g., extend, such as in a concave or convex direction) toward or away from each other and/or in the direction of an opposite (e.g., proximal or distal) end of the device.

In certain versions of the disclosed devices having one or more (e.g., one, two, three, four, or five, etc.) arms having a curvilinear configuration (e.g., an arc configuration), the curved portions of each arm may have one or more (e.g., two or more) radii of curvature. In some variations, all of the radii of curvature for the curved portion of one or more (e.g., one, two, three, four, or five, etc.) arms may be the same or different as all of the radii of curvature for the curved portion of one or more (e.g., one, two, three, four, or five, etc.) other arms. For example, the radii of curvature for the curved portions of one, two, or more arms at a first (e.g., distal) end of a device may be different than the radii of curvature for the curved portions of one, two, or more arms at a second (e.g., proximal) end of the device.

Embodiments of the disclosed tissue securing devices have one or more (e.g., one, two, three, four, or five, etc.) arms and/or the center-most lines thereof of the same and/or different lengths. For example, one, two, three, four, or five, etc. arms of a device may extend the same distance from another portion of the device (e.g., an elongated longitudinal structure) as at least one other arm. Likewise, one, two, three, four, or five, etc. arms of a device may extend different distances from another portion of the device (e.g., an elongated longitudinal structure) compared with at least one other arm. In some embodiments, two or more (e.g., two, three, four, or five, etc.) arms (e.g., two or more arms at a proximal end of a device and/or two or more arms at a distal end of a device) have the same length (e.g., distance between an end of the arm at an elongated longitudinal structure and an opposite end of the arm, such as a free end).

In certain variations of tissue securing devices, one or more (e.g., one, two, three, four, five, etc.) arms (e.g., a proximal arm, such as a proximal deployable arm) at a first end (e.g., a proximal end) are connected directly to (e.g., join) one or more (e.g., one, two, three, four, five, etc.) arms (e.g., a distal arm, such as a distal deployable arm) at a second end (e.g., a distal end).

Tissue securing devices, in various instances, include one or more (e.g., one, two, three, four, or five, etc.) connector structures between one or more (e.g., two, three, four, or five, etc.) arms of a device and another portion of the device (e.g., an elongated linear structure or one or more other arms of a device). Such connector structures may be composed of any of the materials described herein or any combinations thereof. Such connector structures may be composed of any suitable polymer and/or metal and/or adhesive.

Embodiments of the disclosed tissue securing devices include devices or one or more (e.g., one, two, three, four, etc.) portions thereof (e.g. elongated linear structures and/or one or more arms) which are symmetrical with respect to one or more (e.g., one, two, three, or four, etc.) planes or only one or more planes. Such planes may include at least a portion of one or more device portions therein and/or be vertical and/or horizontal planes and/or be transverse planes and/or be cross-sectional planes. For example, one or more (e.g., one, two, three, or four, etc.) arms at a first end (e.g., proximal end) may be symmetrical with respect to one or more other arms at the first end (e.g., proximal end), such as arms extending from the elongated linear structure of the device in an opposite direction. Additionally, one or more (e.g., one, two, three, or four, etc.) arms at a second end (e.g., distal end) may be symmetrical with respect to one or more other arms at the second end (e.g., distal end) such as arms extending from the elongated linear structure of the device in an opposite direction.

The body of a tissue securing device and/or one or more (e.g., one, two, three, four, five, etc.) arms may, in certain aspects, be a single continuous piece of one or more materials, such as any of the materials described herein. In some instances, the body and/or one or more (e.g., one, two, three, four, five, etc.) arms of a tissue securing device may have one portion (e.g., one, two, three, or four or more arms, or a portion therebetween, such as an elongated linear structure) composed of one or more materials (e.g., any of the materials described herein) and at least a second portion (e.g., one, two, three, or four or more arms, or a portion therebetween, such as an elongated linear structure) composed of one or more materials which are different or the same as the materials of the first portion and which may be attached (e.g., laser-welded or adhesively attached) to the materials of the first portion.

Devices as described herein and portions thereof (e.g., one or more arms and/or elongated linear structure) may be fabricated from any convenient material or combination of materials. In some embodiments, a tissue securing device, or one or more portions thereof may include biocompatible materials. The phrase “biocompatible materials” are materials that can be placed on or in living tissue for an extended period of time, such as for a period of 2 days or more, such as 1 week or more, 4 weeks or more, 6 months or more, or 1 year or more, e.g., 5 years or more, up to and including the remaining lifetime or expected remaining lifetime of the subject or more, and not cause a significant adverse (e.g., detrimental to health) reaction (e.g., an immune response) in the tissue or the associated organism. Biocompatible materials may or may not be biodegradable.

Biocompatible materials, as included in the subject devices, can include any suitable biocompatible material, such as a shape memory material, which is described in greater detail below. Biocompatible materials of the subject devices, in some instances, are polymeric materials (e.g., materials having one or more polymers) and/or metallic materials. Such materials may have characteristics of flexibility and/or high strength (e.g., able to withstand significant force, such as a force exerted on it by a tissue within a human body, without breaking and/or resistant to wear) and/or high fatigue resistance (e.g., able to retain its physical properties for long periods of time regardless of the amount of use or environment).

In some embodiments, biocompatible polymeric materials of the subject devices, include, but are not limited to: plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc. In certain embodiments, biocompatible metallic materials of the subject devices, include, but are not limited to: metals and metal alloys, e.g., stainless steel, titanium, tantalum, etc.

In some embodiments, the devices include one or more components (e.g., arms) made of a shape memory material. Shape memory materials are materials (e.g., biocompatible materials) that exhibit the shape memory effect, where the materials that have a temperature induced phase change, e.g., a material that if deformed when cool, returns to its “undeformed”, or original, shape when warmed, e.g., to body temperature. Where desired, the shape memory may be one with a transformation temperature suitable for use with a stopped heart condition where cold cardioplegia has been injected for temporary paralysis of the heart tissue (e.g., temperatures as low as 8-10 degrees Celsius). The shape memory material may also be heat activated, or a combination of heat activation and pseudoelastic properties may be used. Shape memory materials of interest include shape memory metal alloys, such as alloys of nickel (e.g., nickel titanium alloy (NITINOL®), nickel cobalt alloys (e.g., ELGILOY® nickel cobalt chromium alloy, etc.), zinc, copper (e.g., CuZnAl), gold, iron, etc. Also of interest are non-metallic materials that exhibit shaper memory qualities, e.g., shape memory plastics, etc.

In certain embodiments, an active agent may be included in the composition of a biocompatible material, such as a polymeric material. As used herein, the phrase “active agent” refers to one or more chemical substances that, when administered to (e.g., placed in contact with or ingested by) a human, have one or more physiological effects. In some embodiments, the one or more active agents include an antithrombotic substance and/or an antibiotic substance and/or an anti-inflammatory (e.g., a substance that reduces or prevents inflammation). In various embodiments, a tissue securing device may be coated with a polymer, such as a polymer that releases one or more active agents (e.g., an anticoagulant that thereby reduces the risk of thrombus formation).

Embodiments of the subject devices and materials thereof may have one or more flexibilities and/or rigidities. The term “flexible” is used in its conventional sense to mean capable of being bent, usually without breaking, e.g., easily bent. The term “rigid” is also used in its conventional sense to mean stiff or unyielding, e.g., not pliant or flexible, in other words hard. In certain aspects, tissue securing device or one or more portions (e.g., one or more arms and/or an elongated linear structure) or materials thereof is flexible (e.g., the device can be flexed when pressure is applied to the device by a human heart or hand). In certain aspects, a tissue securing device or one or more portions (e.g., one or more arms and/or an elongated linear structure) or materials thereof is rigid (e.g., the device cannot be easily flexed or flexed at all when pressure is applied to the device by a human heart or hand). In certain aspects, a tissue securing device or one or more portions (e.g., one or more arms and/or an elongated linear structure) or materials thereof is semi-rigid.

Further examples of characteristics or applications of tissue securing devices that may be utilized either wholly or partially in connection with the disclosed devices and methods are provided by U.S. Pat. Nos. 6,447,524; 6,425,900; 5,582,611; 7,150,750; 6,113,611; 6,290,702; 6,325,805; 4,627,437; 7,056,330; 8,292,154; 6,074,418; 7,828,187; 8,282,670; 4,261,244; 4,887,601; 4,489,875; 5,366,479; 7,056,333; 5,456,400; 5,964,772; 389,660; and 5,342,376, as well as U.S. Provisional Pat. App. No. 61/831,450 and international application serial no. PCT/US2014/______ having Attorney Docket No. LCTHX-007WO and titled “ANNULOPLASTY DEVICE” and filed on Jun. 4, 2014; as well as U.S. Published Patent Application Nos. 20030097148; 20030229360; 20020117534; the disclosures of each which are incorporated by reference herein.

### Hand-Held Delivery Apparatuses

The disclosed embodiments include delivery apparatuses configured to deliver a tissue securing device to a target tissue site. The terms “delivery”, “deliver” and “delivering”, as used herein, refer to moving an aspect (e.g., a tissue securing device) spatially (e.g., moving the aspect from a first position, e.g., a point outside a subject's body, to a second position, e.g., a point inside a subject's body). Delivery may also include leaving the aspect at the second position, e.g., a point inside a subject's body) for a time period, such as for a period of months, years and/or for at least the remaining lifetime of the subject.

As used herein, the phrase “tissue site” refers to a location within the body of a subject at, within, contacting, above, below, or immediately adjacent to (e.g., within 1, 2, 3, 4, 5, 6, 7, 8, 9, or 10 mm of) one or more tissues (e.g., the annulus of a cardiac valve) to which one or more devices (e.g., one or more tissue securing devices and/or an implantable device) may be secured. For example, a tissue site may be a cardiac valve site (e.g., a mitral valve site). Accordingly, the phrase “target tissue site” refers to a tissue site that is targeted to receive (e.g., receive delivery of) one or more device (e.g., a tissue securing device). Such a site may be targeted, for example, by an operator of a delivery apparatus (e.g., a surgeon) and/or another diagnosing and/or operating professional (e.g., doctor, medical specialist, nurse, etc.). A tissue site, such as a target tissue site, may be a tissue location, as “tissue location” is described herein.

Embodiments of the subject delivery apparatuses include a tissue securing device including a body having a proximal deployable arm at a proximal end and a distal deployable arm at a distal end, wherein upon deployment, the proximal and distal deployable arms are in a non-coplanar configuration. Variations of the disclosed delivery apparatuses may include any of the embodiments of the tissue securing devices described herein.

In some instances, the disclosed delivery apparatuses include one or more (e.g., one, two, three, four, five, etc.) actuators configured to deploy and/or deliver a tissue securing device upon actuation. In certain embodiments, actuators include one or more portions or structures (e.g., handles, elongated structures, levers, gears, slides, biasing members, e.g., springs etc.) of an apparatus which may be interconnected and which actuate (e.g., move, such as along a linear direction and/or by rotating, a distance in a first direction and/or then move the same distance in a second opposite direction) (e.g., concertedly actuate). In various embodiments, actuators include one or more activation portions which are configured to be moved to initiate actuation and/or one or more delivering portions which are configured to deliver a device, such as a tissue securing device. In certain embodiments in which actuators include one or more activation portions, one or more ejectors, which are configured to deliver and/or deploy a device, such as a tissue securing device are included in a portion of the device other than the actuator (e.g., the distal end elongated structure). The term “deploy” is described as it applies to tissue securing devices throughout this application. Furthermore, as used herein, the terms “actuation” or “actuating” may refer to causing one or more portions of an apparatus to move (e.g., move, such as along a linear direction and/or by rotating, a distance in a first direction, e.g., a distal direction or substantially distal direction, and/or then move the same distance in a second opposite direction, e.g., a proximal direction or substantially proximal direction). Actuation may result, for example, from pulling a lever, pushing a button, flipping a switch, sliding a slide-able structure, or any combination thereof. An actuator may also be manually actuated (e.g., actuated as a result of an operator pulling a lever) and/or electronically actuated (e.g., actuated by an electronic control system, such as a control system including a computer). Additionally, apparatuses as disclosed herein and/or portions thereof (e.g., a handle and/or an elongated structure) may also have a distal end and a proximal end.

Various embodiments of the disclosed delivery apparatuses are hand-held apparatuses. As used herein, the term “hand-held” refers to the characteristic ability of an aspect to be held (e.g., retained, or easily or comfortably held) in a hand, such as the hand of a mammal, such as the hand of a human, such as the hand of an adult male or female human of an average size and/or strength. As such, a hand-held aspect is an aspect that is sized and/or shaped to be retained (e.g., easily or comfortably retained) in the hand of a human. A hand-held aspect may also be an aspect that may be moved (e.g., easily moved, such as easily moved in a vertical and/or horizontal direction) by a human (e.g., one or two hands of a human).

In certain versions of the disclosed delivery apparatuses, the apparatuses have an actuator and a proximal end handle including the actuator (e.g., one or more levers and/or buttons on the actuator). In various aspects, handles of the apparatuses are configured to be gripped by one or two hands of a human and/or have a gripping portion. In some instances of the disclosed delivery apparatuses, the apparatuses are shaped as a gun. In such instances, the handle of the apparatus is shaped like the handle of a gun. In some instances, handles of apparatuses have a first end (e.g., an end at which an elongated structure, e.g., a distal end elongated structure of an apparatus, may be attached) and a second end.

In various aspects of the disclosed apparatuses, handles of the apparatuses are shaped as or substantially shaped as a cuboid, a cube, a cylinder, a square based pyramid, a rectangular based pyramid, a cone, a triangular prism, a triangular based pyramid, a sphere, a combination thereof, or another shape. In certain versions of handles of the disclosed apparatuses, a cross section (e.g., a profile of the handle within a plane, such as a cross-sectional plane, that is perpendicular to or substantially perpendicular to the center-most line or a tangent line thereof) of an handle at a point along the handle is shaped as a circle (e.g., forming a circular cross-section), semi-circle, oval, rectangle (e.g., forming a rectangular cross-section), square, triangle, polygon, quadrilateral, or any combination thereof. Such a cross section of a handle may also be curvilinear.

Embodiments of the disclosed apparatuses include apparatuses having a handle having the actuator within (e.g., entirely contained within and/or between at least two portions of) the handle. In various embodiments, an actuator can be at or substantially at a first end and/or a second end of a handle of an apparatus. Some embodiments of the disclosed apparatuses include apparatuses having a handle having a main body portion and an actuator (e.g., an at least partially rotatable actuator or an actuator that actuates in an arc) extending therefrom. Various embodiments of the disclosed apparatuses include apparatuses having a handle having a main body portion and an actuator at a location, contacting, on top of, below, and/or immediately adjacent to the main body portion of the handle.

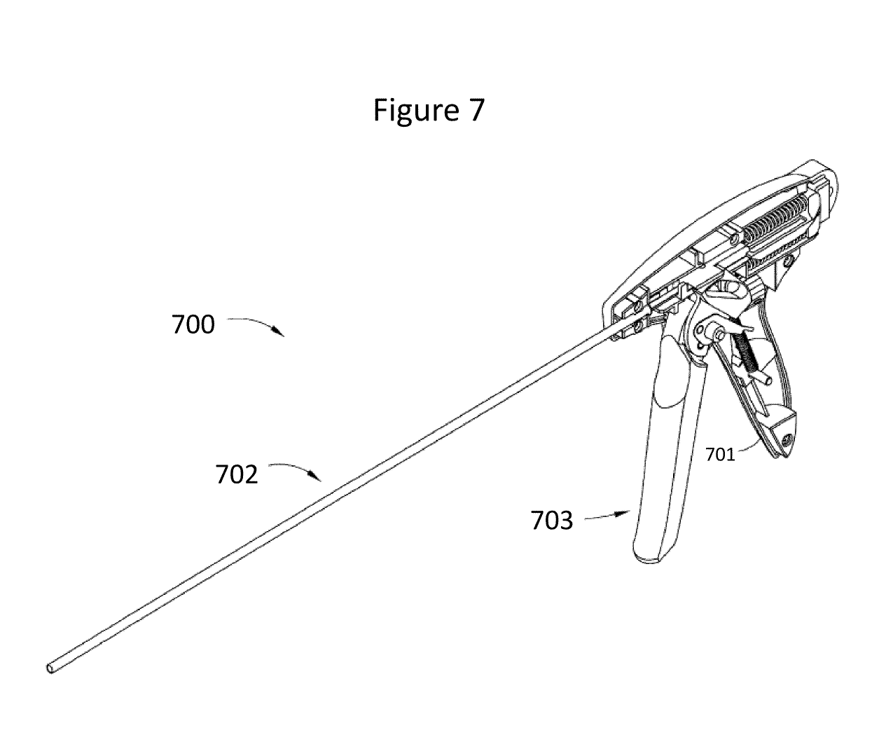

FIG. 7 provides an illustration of an embodiment of a delivery apparatus 700. Delivery apparatus 700 is shown having a handle 701 and a distal end elongated structure 702 extending from the handle 701. An actuator 703 is also shown extending from the handle 701. Actuator 703 is depicted in an un-actuated position.

In certain variations of the disclosed delivery apparatuses, such as versions in which the apparatuses have an actuator and a proximal end handle including the actuator, apparatuses include (e.g., additionally include) a tissue securing device (e.g., a tissue securing device in an un-deployed configuration) and a distal end elongated (e.g., longitudinal) structure including (e.g., housing at least partially therein) the tissue securing device.

As noted above, in some instances of the subject delivery apparatuses, the apparatuses are shaped as a gun. In such instances, the distal end elongated structure of the apparatus is shaped like the portion of a gun extending from the handle (e.g., the barrel portion). In some instances, distal end elongated structures of apparatuses have a first end (e.g., an end, such as a proximal end, at which a handle may be attached) and a second end, such as a distal end.

In various certain aspects of the disclosed apparatuses, distal end elongated structures of the apparatuses are shaped as or substantially shaped as a cuboid, a cube, a cylinder, a square based pyramid, a rectangular based pyramid, a cone, a triangular prism, a triangular based pyramid, a sphere, a combination thereof, or another shape. In some versions, a distal end elongated structure or a portion thereof may also be curvilinear and/or form an arc.

In some aspects, distal end elongated structures may have a center-most line (e.g., a straight line) extending through the center-most portion of the structures (e.g., the center-most points of each transverse, e.g., perpendicular, cross section of the structure along the longitudinal length of the structure). In some aspects, distal end elongated structures may have an axis (e.g., an axis of symmetry and/or a long axis) extending (e.g., extending longitudinally) through and along the length of a distal end elongated structures.

In some embodiments of apparatuses including distal end elongated structures, the diameter, radius, thickness, peripheral circumference and/or cross sectional area (e.g., the area of the structure within a plane, such as a cross-sectional plane that is perpendicular to or substantially perpendicular to the axis) of a distal end elongated structure along the length of its axis remains the same. In some embodiments, such dimensions increase and/or decrease along the length of the axis of a distal end elongated structures from a first to a second end of the distal end elongated structure or from a second end to a first end of the distal end elongated structure.

In certain versions of distal end elongated structures of the disclosed apparatuses, a cross section (e.g., a profile of the distal end elongated structure within a plane, such as a cross-sectional plane, that is perpendicular to or substantially perpendicular to the center-most line or a tangent line thereof) of a distal end elongated structures at a point along the structure is shaped as a circle (e.g., forming a circular cross-section), semi-circle, oval, rectangle (e.g., forming a rectangular cross-section), square, triangle, polygon, quadrilateral, or any combination thereof. Such a cross section of a distal end elongated structure may also be curvilinear.

In some versions of the apparatuses, the handle and the distal end elongated structure may be attached (e.g., adhesively, snap-edly, etc.) at the ends of each aspect where they contact each other. In some variations, a portion of a handle fits within a distal end elongated structure and/or a portion of a distal end elongated structure fits within a handle.

In some variations of the disclosed delivery apparatuses, the distal end elongated structure includes (e.g., houses) one or more tissue securing devices at a first (e.g., distal) and/or second (e.g., proximal) end. In certain variations of the disclosed delivery apparatuses, the distal end elongated structure is configured to deploy one or more tissue securing devices at or from a first (e.g., distal) end. In certain versions of the disclosed delivery apparatuses, the distal end elongated structure includes (e.g., retains) one or more (e.g., one, two, three, four, five, six, seven, eight, nine, ten, etc.) tissue securing devices at a location, within, contacting, on top of, below, and/or immediately adjacent to the distal end elongated structure or a portion thereof (e.g., a main body portion or a proximal and/or distal end of the distal end elongated structure).

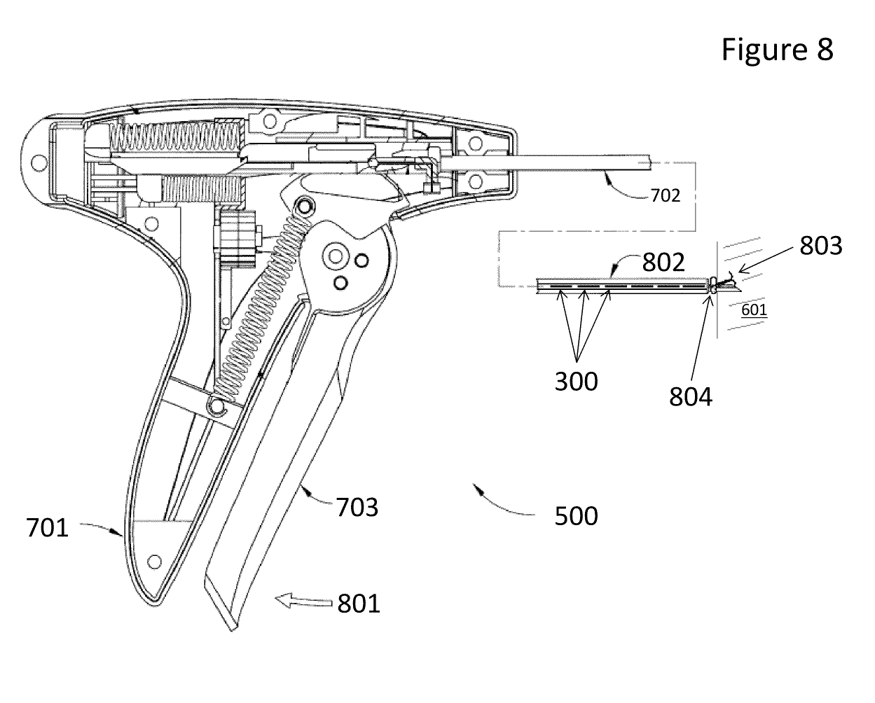

FIG. 8 provides an illustration of a delivery apparatus 700. FIG. 8 specifically shows the handle 701 of the apparatus having an actuator 703 extending therefrom. Actuator 703 is shown in an actuated position following actuation in direction 801. Also depicted is a distal end elongated structure 702 including a partial cross-section 802 thereof. The partial cross-section 802 of the distal end elongated structure 702 shows tissues securing devices 300 in an un-deployed configuration within the elongated structure. Also illustrated is a tissue securing device 803 deploying into tissue 601. Shown adjacent to deploying tissue securing device 803 is a portion of an aspect 804, such as a tissue or prosthetic device, being affixed to tissue 601 with tissue securing device 803.

In certain embodiments, the distal end elongated structure of the delivery apparatus or a portion thereof is configured (e.g., sized and/or shaped) to access an internal tissue site (i.e., a tissue site within the body of a subject). For example, in some variations, the diameter or one or more transverse cross sectional thicknesses of the distal end elongated structure along the longitudinal length of the structure (e.g., at the distal end of the structure) may range from 1 mm to 10 mm, such as 2 mm to 5 mm or 1 mm to 3 mm. Additionally, in some variations, the diameter or one or more transverse cross sectional thicknesses of the distal end elongated structure along the longitudinal length of the structure (e.g., at the distal end of the structure) may be 3 mm or less.

Embodiments of the subject apparatuses include apparatuses wherein the distal end elongated structure is configured to access an internal tissue site by a minimally invasive protocol. As used herein, the phrase “minimally invasive protocol” refers to a protocol that is less invasive than an open surgical procedure which is described in greater detail below, and/or is used for the same purpose as an open surgical procedure. A minimally invasive protocol may involve the use of arthroscopic and/or laparoscopic and/or thoracoscopic devices and/or remote-control manipulation of surgical instruments.

In certain embodiments of the disclosed apparatuses, a minimally invasive protocol is an endoscopic protocol. As used herein, the phrase “endoscopic protocol” refers to a protocol performed using one or more scopes (e.g., an endoscope) going through one or more body openings. An endoscopic protocol may be a minimally invasive surgical procedure, as is described in further detail below. Also, an endoscopic protocol (e.g., an endoscopic procedure) or percutaneous protocol (e.g., an endovascular percutaneous catheter procedure) may be used to treat and/or diagnose disease.

Embodiments of the subject apparatuses include apparatuses wherein the distal end elongated structure or a portion thereof (e.g., a distal-end portion) is articulating. As used herein, the term “articulating” refers to the ability to be moved (e.g., oriented) in more than one direction. In variations of the apparatuses having a distal end elongated structure or a portion thereof (e.g., a distal-end portion) that is articulating, the articulating portion may be configured to be oriented to deploy a tissue securing device effectively (e.g., in a manner wherein the tissue securing device enters tissue in a direction that is perpendicular to the surface of the tissue at that location).

In various embodiments of the apparatuses, the disclosed embodiments include, carry, or house two or more (e.g., three, four, five, etc.) tissue securing devices. In such embodiments, one or more of the two or more tissue securing devices may be within (e.g., prior to deployment) the handle portion of an apparatus and/or within the distal end elongated structure of the apparatus. In some embodiments, apparatuses are configured so that two or more (e.g., three, four, five, etc.) tissue securing devices may be contacting each other within the device.

Additionally, supplementary examples of devices (e.g., apparatuses), and/or methods or characteristics thereof that may be utilized either wholly or partially in connection with the disclosed devices (e.g., apparatuses) and/or methods are provided by U.S. Provisional Patent Application No. 61/831,450 and international application serial no. PCT/US2014/______ having Attorney Docket No. LCTHX-007WO and titled “ANNULOPLASTY DEVICE” and filed on Jun. 4, 2014, and U.S. Pat. No. 6,425,900, the disclosures of each which are incorporated by reference herein.

### Methods

The subject devices find use in methods for securing tissue. In certain embodiments, the methods include a step (e.g., a sequential step and/or simultaneous step with another step, such as a step of positioning a device) of deploying a tissue securing device, such as any of the tissue securing devices described above.

As used herein, the term “deploying” refers to causing or allowing a device (e.g., a deployable device) and or one or more portions (e.g., deployable portions) thereof (e.g., one, two, three, four, or more arms) to deploy. More specifically, and as described above, in some aspects, tissue securing devices may be un-deployed (i.e., have an un-deployed configuration) when one or more (e.g., two, three, four, five, etc.) arms of the device are un-deployed. Likewise, in some aspects, tissue securing devices may be deployed (i.e., have a deployed configuration) when one or more (e.g., two, three, four, five, etc.) arms of the device are deployed.

The disclosed methods, in certain variations, include methods wherein tissue securing devices are deployed at a tissue location. As used herein, the phrase “tissue location” refers to a location at, contacting, on top of, below, and/or immediately adjacent to (e.g., within 1, 2, 3, 4, 5, 6, 7, 8, 9, or 10 mm of) one or more tissues (e.g., the annulus of a cardiac valve) to which a device (e.g., an annuloplasty device) may be attached. In some embodiments, a tissue location is an internal location (e.g., a location within the body of a subject). In some embodiments, a tissue location (e.g., an internal location) is a cardiac location (e.g., a location within, at, contacting, on top of, below, and/or immediately adjacent to, (e.g., within 1, 2, 3, 4, 5, 6, 7, 8, 9, or 10 mm of) the heart of a subject. In certain embodiments, a tissue location (e.g., an internal location, such as a cardiac location) is a cardiac valve location (e.g., a mitral valve location). As used herein, the phrase “cardiac valve location” refers to the location within the body of a subject at, within, contacting, on top of, below, and/or immediately adjacent to (e.g., within 1, 2, 3, 4, 5, 6, 7, 8, 9, or 10 mm of) one or more cardiac valves (e.g., the mitral, tricuspid, aortic and/or pulmonary valves). Accordingly, the phrase “mitral valve location” refers to the location within the body of a subject at, within, contacting, on top of, below, or immediately adjacent to the mitral valve.

In certain aspects of the methods, the tissue securing device includes a body having one or more (e.g., one, two) proximal deployable arms at a proximal end and one or more (e.g., one, two) distal deployable arms at a distal end, wherein upon deployment, the proximal and distal deployable arms are in a non-coplanar configuration (i.e., a non-coplanar configuration with respect to one another).

In some instances, the disclosed methods include a step (e.g., a sequential step and/or a simultaneous step and/or a step that precedes another step, such as a step of deploying a tissue securing device) of positioning an implantable device at a tissue location. As used herein, the term “positioning” refers to moving an aspect spatially (e.g., moving the aspect from a first point in space, e.g., a point outside a subject's body, to a second point in space, e.g., a point inside a subject's body).

Any of the embodiments of the disclosed implantable devices may be configured (e.g., sized and/or shaped) to be implanted at a tissue location, such as a cardiac valve location (e.g., a mitral valve location). Implantable devices, according to embodiments of the methods, may include an annuloplasty device and/or an annuloplasty ring and/or an implantable prosthetic, such as a mesh (e.g., a hernia mesh). Examples of devices (e.g., implantable devices), or applications or characteristics thereof that may be utilized either wholly or partially in connection with the disclosed devices and methods are provided by U.S. Provisional Patent Application No. 61/831,450 and international application serial no. PCT/US2014/______ having Attorney Docket No. LCTHX-007WO and titled “ANNULOPLASTY DEVICE” and filed on Jun. 4, 2014; as well as U.S. Pat. Nos. 5,104,407; 6,425,900 and 6,447,524; the disclosures of each which are incorporated by reference herein.

As noted above, the term “deploying”, as used herein, refers to causing or allowing a device (e.g., a deployable device) and or one or more portions (e.g., deployable portions) thereof (e.g., one, two, three, four, or more arms) to deploy. In some embodiments of the methods, deploying includes associating one or more (e.g., one, two, three, four, five, etc.) tissue securing devices with one or more (e.g., one, two, three, four, five, etc.) implantable devices and tissue at a tissue location in a manner sufficient to stably associate the implantable device with the tissue location. As used herein, the term “associating” refers to positioning one or more aspects (e.g., devices) in proximity (e.g., a close, adjacent, abutting, contacting and/or at least partially enveloping proximity). In some variations, associating may include piercing, and/or introducing at least a portion of an aspect (e.g., a tissue securing device) into another aspect (e.g., a tissue). Accordingly, by “stably associating” is meant that the aspect (e.g., the device) is substantially if not completely fixed relative to the tissue location of interest. For example, a tissue securing device and/or an implantable device, that is stably associated with a tissue in the body of a subject may be retained at the same position or substantially at the same position as the tissue within the body of a subject for a time period, such as a for a period of months, years and/or for at least the remaining lifetime of the subject.

In various embodiments of the methods, deploying includes heating a tissue securing device (e.g., heating the device to an internal human body temperature). By heating such a device that is composed of a shape memory material, the material may change its shape (e.g., change its shape to assume a deployed configuration). Heating a tissue securing device may include implanting the device into living tissue.

In some embodiments of the subject methods, the methods include securing and/or associating one tissue to or with another tissue (e.g., securing by deploying one or more, e.g., one, two, three, four, etc., proximal arms of a tissue securing device within tissue and deploying one or more distal arms of a tissue securing device within tissue). In some versions, the embodiments of the methods include securing and/or associating a device (e.g., an implantable device) to or with a tissue (e.g., securing by deploying one or more proximal arms of a tissue securing device within tissue and deploying one or more distal arms of a tissue securing device at least partially around a portion of an implantable device, e.g., such that at least one portion of the implantable device lies between at least two portions of the tissue securing device).

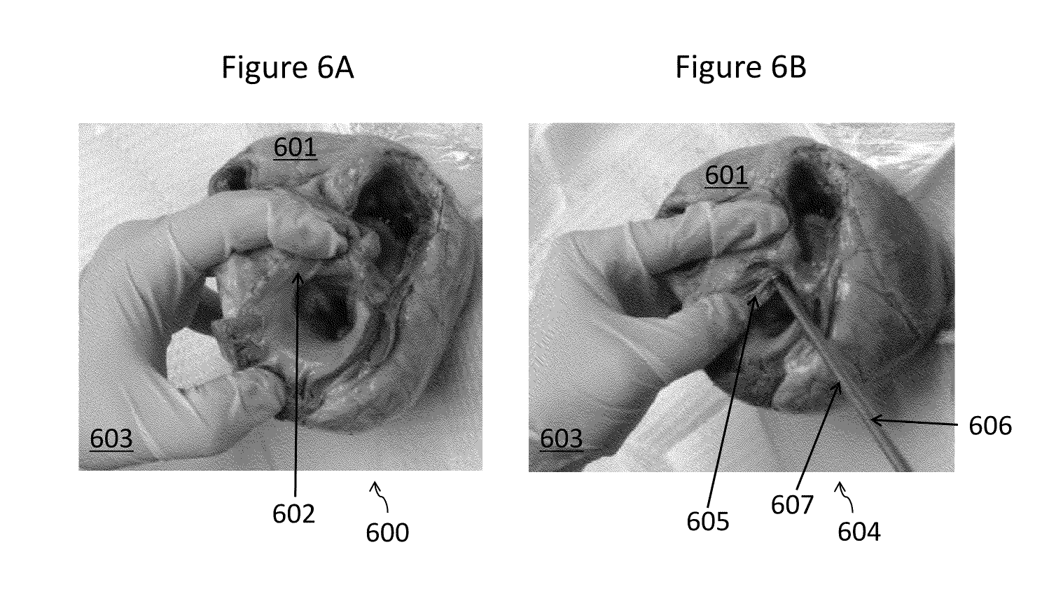

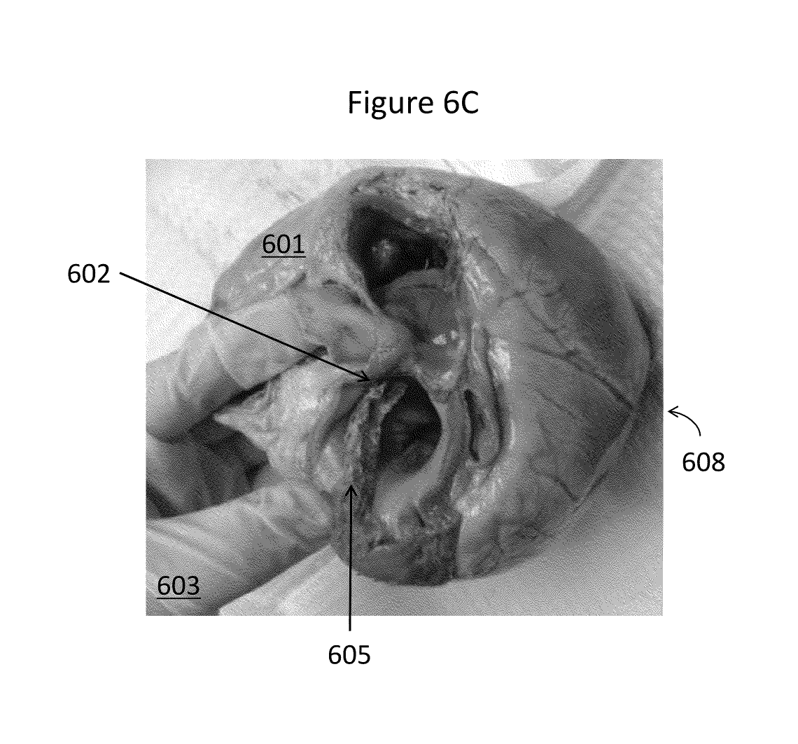

FIGS. 6A-6C illustrate steps for fastening an implantable device to a tissue according to embodiments of the subject methods. Specifically, FIG. 6A provides a depiction of a tissue 601 (e.g., heart) site (e.g., a target tissue site) 602 being exposed 600. FIG. 6A also illustrates an operator 603. A step of fastening 604 an implantable device 605 to a tissue 601 is shown in FIG. 6B. Included in FIG. 6B is a view of a tissue 601 (e.g., heart) site (e.g., a target tissue site) 602. An implantable device 605 is also shown in FIG. 6B as well as a delivery apparatus 606 for delivering a tissue securing device and a distal elongated structure 607, or a portion thereof, of the delivery apparatus 606. FIG. 6B also illustrates an operator 603. Provided in FIG. 6C is a view 608 of an implantable device 605 secured (e.g., secured with a tissue securing device) at a tissue 601 (e.g., heart) site (e.g., a target tissue site) 602. FIG. 6C also shows an operator 603.

The devices used in accordance with the disclosed methods may be composed of a wide variety of one or more materials, such as the biocompatible materials listed herein. As noted above, one or more portions (e.g., one or more arms and/or elongated linear structure) of the disclosed devices include one material or a variety of materials. In some embodiments, the tissue securing devices used in accordance with the disclosed methods or one or more portions thereof (e.g., one or more arms and/or elongated linear structure) include a metal (e.g., stainless steel). In certain variations, the tissue securing devices used in accordance with the disclosed methods or one or more portions thereof (e.g., one or more arms and/or elongated linear structure) include a material that is a shape memory material.

In certain versions of the disclosed methods, the method is a surgical procedure. As used herein, the phrase “surgical procedure” refers to a procedure (e.g., a medical procedure) involving at least one incision in the body of a subject and/or performed using one or more instruments (e.g., surgical instruments). A surgical procedure may be carried out through a body cavity and/or through the skin of a subject.

As noted above, in certain variations of the disclosed methods, the method is an open surgical procedure. As used herein, the phrase “open surgical procedure” refers to a surgical procedure wherein at least one long incision (e.g., having a length of 10 cm) is made in the body of a subject to introduce at least one surgical instrument and/or visualize the surgery through the incision. In an open surgical procedure, closure devices, e.g., staples, sutures, etc., may be used to close at least one incision.

In certain variations of the disclosed methods, the method is a minimally invasive surgical procedure. As used herein, the phrase “minimally invasive surgical procedure” refers to a surgical procedure that is less invasive than an open surgical procedure. A minimally invasive surgical procedure may involve the use of arthroscopic and/or laparoscopic devices and/or thoracoscopic devices and/or remote-control manipulation of surgical instruments. Minimally invasive procedures include endovascular procedures, which may be totally endovascular procedures, percutaneous endovascular procedures, etc. Endovascular procedures are procedures in which at least a portion of the procedure is carried out using vascular access, e.g., arterial access.

In some versions of the methods, one or more tissue securing devices are present in or on a hand-held delivery apparatus (e.g., any of the hand-held delivery apparatuses described herein) and deploying includes actuating the hand-held delivery apparatus to deploy the one or more tissue securing devices, for example, to a target tissue site.

### Utility

The devices and methods of the invention, e.g., as described above, find use in a variety of different applications, e.g., applications where attaching one tissue to another tissue or attaching a device (e.g., an implantable prosthetic device) to tissue within the body of a patient is desired. As such, the devices and methods of invention find use in the closing a cut or a wound by holding tissues on opposing sides of the cut or wound together. Using the subject devices and methods in such a procedure may facilitate healing of the cut or wound. The devices and methods as disclosed herein also find use in surgical procedures in which one tissue is attached to another tissue within the body of a subject. In such as a procedure, e.g., a fundoplication procedure to treat gastroesophageal reflux disease (GERD), tissue securing devices may be applied to attach a first tissue to a second tissue.

Additionally, the subject devices and methods may be used in a procedure in which a device is introduced into the body of a patient and attached with a surgical fastener to tissue within the body of a patient. For example, the tissue securing devices as described herein may be used in the implantation of surgical mesh material to attach the mesh material to tissue. The attached surgical mesh may, in turn support, immobilize and/or position one or more one or more body organs or other tissues during surgery or be used for the treatment of urinary incontinence, uterine prolapse or hernia.

Another example of a procedure in which the subject devices and methods find use in an annuloplasty procedure, in which an annuloplasty device, e.g., ring, is stably associated with a cardiac location, such as a valve location (e.g., the mitral valve) in order to improve function of the valve. Such procedures are further described in U.S. Provisional Patent Application No. 61/831,450 and international application serial no. PCT/US2014/______ having Attorney Docket No. LCTHX-007WO and titled “ANNULOPLASTY DEVICE” and filed on Jun. 4, 2014, the disclosure of which is incorporated by reference herein.

The devices and methods which are disclosed herein may be configured to be used to engage a substantial volume of tissue with, for example two arms on a distal end, and to thereby effectively attach one end (e.g., the distal end of the device) with the tissue. The devices and methods which are disclosed herein may also configured to engage a substantial volume of tissue or a prosthetic device with, for example two arms on a proximal end, and to thereby effectively attach one end (e.g., the proximal end of the device) with the tissue or prosthetic device.

Furthermore, the tissue securing devices of the subject invention may also be configured (e.g., have a shape) to be removed from tissue following their deployment without causing substantial damage to surrounding tissue. For example, embodiments of tissue securing devices as disclosed herein may be removed from tissue without cutting or tearing the tissue.

In addition, the disclosed subject matter provides devices, apparatuses and methods to implant such devices in a time-efficient manner. For example, attaching tissues or implantation of a prosthetic device, including measuring the dimensions of the relevant tissue site and anchoring (e.g., suturing), a prosthetic device at the site, can be a time-consuming process. In addition, attaching tissues or introducing and/or anchoring a prosthetic device to tissue is often technically difficult and/or time-consuming when using minimally invasive procedures because of limitations in using 2-dimensional video for viewing the surgical field, limited exposure of the surgical field, and/or limited degrees of freedom when using standard instrumentation (e.g., thoracoscopic instrumentation).

By using the subject devices, apparatuses and methods, such as using tissue securing devices and apparatuses to deliver them at a tissue site, the total time that a procedure (e.g., attachment of tissues or a prosthetic implantation procedure) takes can be reduced. The time of such a procedure can be reduced by eliminating or reducing the time required for attaching tissues or suturing the prosthetic device to a tissue site. A reduced time for a surgical process can help prevent fatigue in attending medical staff and can otherwise reduce risk to the patient.

Additionally, by applying the described devices, apparatuses and methods in securing a tissue to another tissue or a prosthetic device to a tissue site, the total trauma incurred by a tissue at the relevant site (e.g., the site of implantation) may be reduced. For example, by applying the disclosed devices, apparatuses and methods, the need for sutures an associated trauma in attaching a prosthetic device to a tissue site can be reduced or eliminated.

Furthermore, by applying the disclosed devices, apparatuses and methods, such as using tissue securing devices in securing a tissue to another tissue or a prosthetic device to a tissue site, the securing process can be significantly simplified (e.g., made easier to understand) and/or made easier to or perform. For example, the process may be simplified by reducing or eliminating the amount of sutures (e.g., sutures at the tissue site) needed for attachment of tissue or implantation of the device. A process of attaching tissues or prosthetic device implantation may also be simplified by reducing the total steps performed using a 2-dimensional video for viewing the surgical field, while having a limited exposure of the surgical field, and/or while having limited degrees of freedom by using standard instrumentation (e.g., thoracoscopic instrumentation).

### Kits

Also provided are kits that at least include the subject devices and which may be used according to the subject methods. The subject kits at least include an implantable device and a tissue securing device.

In certain embodiments, the disclosed kits include a tissue securing device including a body having a proximal deployable arm at a proximal end and a distal deployable arm at a distal end, wherein upon deployment the proximal and distal deployable arms are in a non-coplanar configuration. Other tissue securing devices that may be included in the subject kits may be any of the tissue securing devices described or referred to herein.

Embodiments of the subject kits include an implantable device, such as an annuloplasty device. The term “annuloplasty”, as used herein, refers to a surgical procedure in which a heart valve (e.g., mitral valve) annulus is remodeled. Remodeling a heart valve may include, for example, reinforcing and/or re-shaping the valve by implanting (e.g., introducing and attaching to tissue) an annuloplasty device, such as a prosthetic ring (e.g., “annuloplasty ring”) to stabilize the annulus and to correct or prevent valvular insufficiency.

In various embodiments of the disclosed kits, annuloplasty devices included therein include an at least partially annular body and a tissue securing region integrated into and of differing composition from the body, where the tissue securing region is configured to receive a tissue securing device. The term “annular” as used herein refers to the characteristic of forming or being shaped like a ring (e.g., shaped generally like a torus). Such a ring shape may be full (e.g., forming a continuous band of material) or partial (e.g., forming a portion of a continuous band of material to form, for example, half of a generally circular shape). An annular aspect may also be circular or ovoid and have a cross section at any or all portions along its length having any shape (e.g., circular, ovoid, rectangular, etc.). An annular aspect may have one or more (e.g., two, three, or four) peripheral edges that form a curve. An annular aspect, in certain aspects, can also form an ellipse and/or may have one or more (e.g., two, three, or four) peripheral edges having straight (e.g., non-curved) portions.

Additionally, the phrase “tissue securing regions” of annuloplasty devices, as used herein, refers to regions of the devices configured for use in attaching the annuloplasty device to tissue of a subject. Attaching an annuloplasty device to tissue of a subject may include securing (e.g., affixing, fastening, anchoring or attaching) the annuloplasty device to the tissue for a period of time (e.g., for the remaining lifetime of a subject or for a portion of the remaining expected lifetime of a subject).

In various aspects, tissue securing regions of annuloplasty devices are integrated into the body of the annuloplasty devices. For example, in some aspects, tissue securing regions may include one or more voids (e.g., holes) in the annuloplasty device (e.g., holes through the body of an annuloplasty device, such as holes which are sized and/or shaped to receive a tissue securing device). In some aspects, one or more tissue securing regions may be fully or partially contained within (e.g., positioned between two portions of) the body of an annuloplasty device. Tissue securing regions or portions of tissue securing regions of annuloplasty devices may also follow the same general contour of the body of the annuloplasty device.

The disclosed kits may also include any of the implantable devices, such as annuloplasty devices, or characteristics thereof which are provided by U.S. Provisional Patent Application No. 61/831,450 and international application serial no. PCT/US2014/______ having Attorney Docket No. LCTHX-007WO and titled “ANNULOPLASTY DEVICE” and filed on Jun. 4, 2014, the disclosure of which is incorporated by reference herein.

Embodiments of the subject kits include kits wherein one or more tissue securing devices are present in (e.g., within, such as housed within, or contained) a delivery apparatus, such as any of the delivery apparatuses described herein.

The tissue securing devices of the subject kits may be composed of a wide variety of one or more materials. As noted above, one or more portions (e.g., one or more arms and/or elongated linear structure) of the disclosed devices include one material or a variety of materials (e.g., the biocompatible materials listed herein). In some embodiments, the tissue securing devices or one or more portions thereof (e.g., one or more arms and/or elongated linear structure) include a metal (e.g., stainless steel). In certain variations, the tissue securing devices or one or more portions thereof (e.g., one or more arms and/or elongated linear structure) include a material that is a shape memory material, such as any of the shape memory materials described herein.

In various embodiments, the kits which are disclosed herein include instructions, such as instructions for using devices and/or apparatuses. The instructions for using devices are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g., portable flash drive, CD-ROM, diskette, etc. The instructions may take any form, including complete instructions for how to use the device or as a website address with which instructions posted on the world wide web may be accessed. Any of the components may be present in containers or packaging, where two or more components may be present in the same container, e.g., as desired. In some instances, the containers/packaging are sterile, e.g., to maintain the sterility of the components of the kit, such as the components that are ultimately to be implanted into a patient.

In addition, embodiments of the disclosed kits or their components may be used according to any of the embodiments of the methods described herein or combinations thereof.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An implantable tissue securing device comprising:
a body having a proximal deployable arm at a proximal end and a distal deployable arm at a distal end, wherein upon deployment the proximal and distal deployable arms are in a non-coplanar configuration.

2. The tissue securing device according to claim 1, wherein the tissue securing device comprises first and second proximal deployable arms at the proximal end of the body.

3. The tissue securing device according to claim 2, wherein upon deployment the first and second proximal deployable arms are in a planar configuration.

4. The tissue securing device according to claim 1, wherein the tissue securing device comprises first and second distal deployable arms at the distal end of the body.

5. The tissue securing device according to claim 4, wherein upon deployment the first and second distal deployable arms are in a planar configuration.

6. The tissue securing device according to claim 1, wherein the proximal and distal deployable arms deploy orthogonally from the body.

7. The tissue securing device according to claim 1, wherein upon deployment, the proximal and distal deployable arms assume a curvilinear configuration.

8. The tissue securing device according to claim 7, wherein the curvilinear configuration is an arc configuration.

9. The tissue securing device according to claim 1, wherein the body comprises an elongated linear structure.

10. The tissue securing device according to claim 9, wherein the elongated linear structure comprises a rectangular cross-section.

11. The tissue securing device according to claim 9, wherein the elongated linear structure comprises a curvilinear cross-section.

12. The tissue securing device according to claim 11, wherein the elongated linear structure comprises a circular cross-section.

13. The tissue securing device according to claim 1, wherein the tissue securing device comprises a metal.

14. (canceled)

15. The tissue securing device according to claim 1, wherein the tissue securing device comprises shape memory material.

16. The tissue securing device according to claim 15, wherein the shape memory material comprises a metal alloy.

17-19. (canceled)

20. The tissue securing device according to claim 1, wherein the body has a length ranging from 1 to 30 mm.

21. The tissue securing device according to claim 1, wherein the proximal and distal deployable arms each have a length ranging from 1 to 10 mm.

22. A method comprising:
deploying a tissue securing device at a tissue location, wherein the tissue securing device comprises an implantable body having a proximal deployable arm at a proximal end and a distal deployable arm at a distal end, wherein upon deployment the proximal and distal deployable arms are in a non-coplanar configuration.

23-37. (canceled)

38. A delivery apparatus configured to deliver a tissue securing device to a target tissue site, the delivery apparatus comprising:
a tissue securing device comprising a body having a proximal deployable arm at a proximal end and a distal deployable arm at a distal end, wherein upon deployment the proximal and distal deployable arms are in a non-coplanar configuration; and
an actuator configured to deploy the tissue securing device upon actuation.

39-45. (canceled)

46. A kit comprising:
(a) an implantable device; and
(b) a tissue securing device comprising a body having a proximal deployable arm at a proximal end and a distal deployable arm at a distal end, wherein upon deployment the proximal and distal deployable arms are in a non-coplanar configuration.

47-56. (canceled)
